(19) United States
(12) Patent Application Publication
(10) Pub. No.: US 2012/0165675 A1
(43) Pub. Date: Jun. 28, 2012

(54) Method For Diagnosis And Treatment Of Vessel Occulsion

(76) Inventor: Paul David Syme, Roxburghshire (GB)

(21) Appl. No.: 10/557,550

(22) PCT Filed: May 21, 2004
(86) PCT No.: PCT/GB04/02207

§ 371 Date: Jan. 31, 2007

(30) Foreign Application Priority Data
May 21, 2003 (GB) 0311667.0
Jun. 6, 2003 (GB) 0313022.6

(51) Int. Cl. A61B 8/06

(52) U.S. Cl. 600/454

(57) ABSTRACT

A non-invasive method of diagnosing and treating vessel occlusion and, in particular, small vessel occlusion by using transcranial Doppler ultrasound scanning. The method can be used for all forms of small vessel ischaemia, including all sub-types of ischaemic stroke, ischaemia secondary to primary intracerebral hemorrhage and intracerebral tumour, diagnosis and treatment is carried out by the identification of abnormal signals during ultrasound intonation.

Method for diagnosis and treatment of vessel occlusion The present invention relates to apparatus and a method for diagnosing and treating small vessel disease using ultrasound technology, and in particular, all sub-types of ischaemic stroke, ischaemia secondary to primary intracerebral hemorrhage and intracerebral tumour.

A stroke occurs when a blood vessel or artery is blocked by a blood clot, thereby interrupting the flow of blood to an area of the brain. Interruption of blood flow to the area of the brain results in cell and neuronal death. The area of dead cells is commonly referred to as an infarct. When brain cells in the infarct die, they are believed to release chemicals that set off a chain reaction of surrounding cell damage sometimes known as “ischeamic cascade.”

Strokes are typified by the loss of function such as speech, movement, vision and memory. When brain cells die, there is a loss of control of abilities which that area of the brain once controlled. For this reason, stroke is a devastating illness. It is ranked number three as a cause of mortality in the Western world and is the main cause of acute severe disability among adults. 5% of the total UK National Health Service budget is spent on treating stroke each year.

Without prompt medical treatment a large area of brain cells can die as the ischeamic progresses at a rapid pace. A critical factor in the treatment of strokes is that the “window of opportunity” for interventional treatment between the vascular event and irreversible neuronal loss is short. Beyond this window, reestablishment of blood flow and administration of neuroprotective agents may have limited effect and in addition can potentially cause further damage or induce side effects. However treatment for stroke in the acute phase is extremely limited. Current treatment includes the administration of aspirin which has an antiplatelet effect. However, the number of patients needed to treat to prevent one stroke is 100, and 1 in 100 can develop haemorrhagic complications.

Thrombolysis refers to the clinical administration of fibrinolytic agents which lyse or dissolve clots. These 25° mimic and assist the endogenous fibrinolysis system in the human body. Blood clots are amorphous in character consisting of a diffuse fibrin meshwork in which blood cells are trapped. The conversion of fluid blood to a solid clot occurs as a result of a complex enzyme cascade which ultimately converts the soluble substance fibrinogen to insoluble strands of fibrin. Thrombolysis^{1 2} given within 3 hours of onset of ischaemic stroke confirmed by scanning may improve outcome among the very few patients who receive this therapy. However statistics suggest that it would be necessary to treat 8 patients within this 3 hour window in order to obtain 1 successful result. Treatment with thrombolysis from 3 to 6 hours after ischaemic stroke is not considered beneficial and may in fact be dangerous as hemorrhages typically occur in 1 in every 26 patients. Whilst promising, thrombolysis is still subject to ongoing trials and a safer alternative would be welcomed. The option of using neuroprotective agents are also subject to ongoing trials³, as previous substances tried have not been proven clinically beneficial. The results of attempts to develop other drugs such as calcium channel and NMDA antagonists to improve the outcome after strokes have so far been disappointing.

Transcranial Doppler ultrasound scanning was invented by Rune Aaslid over 20 years ago. The doppler principle in sonography is based on the insonation of a vessel with an ultrasound signal. This is reflected and backscattered from moving objects (e.g blood cells) with a positive or negative frequency shift. The frequency shift is also called Doppler shift or Doppler signal. The faster the blood cells are moving the higher the Doppler shift. Its use has been of some assistance in the diagnosis of stroke and in the localisation of arterial blockage due to thromboembolism.⁴. It has also been used to monitor vasospasm associated with subarachnoid hemorrhage. Over the last few years, low frequency ultrasound has been shown to increase clot binding and penetration of tissue plasminogen activator (tPA) resulting in increased clot breakdown in vitro. Recently, there is some evidence to support the additive benefit of low frequency ultrasound given in conjunction with recombinant tPA (rtPA) in both coronary arteries and intracerebral arteries. However, the publicly understood theory of the action of ultrasonography and recanalisation relates only to large arteries, and is based on use together with rtPA. Until present, small vessel disease has never been diagnosed using ultrasound. Its use as a therapeutic tool in isolation, has been explored in experimental animals⁵ and in humans.⁶

Currently cases of large vessels being opened using ultrasound in combination with the administration of tissue plasminogen activator (tPA) in combination with ultrasound has been recorded. However there are also known cases of large vessels reopening spontaneously with no improvements in the symptoms of stroke. To date, increased spontaneous recanalisation of large intracerebral arteries has not been shown to produce clinical benefit. However it is known that opening and recanalisation of arteries will limit neurological damage to the benefit of the patient.⁷

The present invention acknowledges and addresses the problems inherent in current methods of diagnosing and treating small vessel ischaemia and disease, and uses ultrasound insonation therapy for all types of ischeamic stroke, ischaemia secondary to primary intracerebral hemorrhage and intracerebral tumour.

It is an aim of at least one aspect of the invention to provide a method of diagnosing vessel disease. In particular it is an aim of at least one aspect of the present invention to provide a method of diagnosing small vessel disease. It is an associated aim to provide a method for screening patients for small vessel occlusive disease. It is a further aim of at least one aspect of the invention to provide a method of targeting and identifying areas of vessel disease with improved accuracy, speed, and effectiveness.

It is a further aim of at least one aspect of the invention to provide apparatus for diagnosing and treating all forms of small vessel ischaemia and damage.

It is also an aim of at least one aspect of the present invention to provide a method for diagnosing and treating large vessel occlusion.

It is a further aim of at least one aspect of the invention to provide an improved method of therapy for treating the symptoms of vessel occlusion.

It is a yet further aim of at least one aspect of the invention to provide an improved method of therapy for all sub-types of stroke and ischaemia secondary to hemorrhage and tumour.

According to a first aspect of the present invention, there is provided the use of ultrasound for the detection of vessel occlusion in a patient.

According to a second aspect of the present invention there is provided a method of diagnosing vessel occlusion in a patient by the use of transcranial doppler ultrasonograhy. Optionally the vessels are small blood vessels.

Alternatively the vessels are large blood vessels.

A diagnostic transcranial Doppler ultrasound machine is used. The ultrasound machine will comprise a display for displaying the signal produced in response to ultrasound. Preferably an ultrasound probe of 2 MHz or less is used.

Preferably diagnosis of the vessel occlusion is carried out by the identification of abnormal ultrasound arterial signals. The abnormal ultrasound arterial signals are found at the baseline within the +/−300 Hz range.

Typically the abnormal ultrasound arterial signals are associated with each cardiac cycle and have an intensity which varies according to the rhythm of the patient's heartbeat.

In small vessels the abnormal ultrasound arterial signals typically resemble the short peak systolic wave and diastolic reversal of flow which can be seen with circulatory arrest due to brain death and are high intensity, low velocity signals.

The abnormal ultrasound arterial signals can be seen at the beginning of each systole. The abnormal ultrasound arterial signal may also have a less obvious diastolic component.

In moderate to larger vessels, the abnormal ultrasound arterial signals may resemble a first harmonic signal. These abnormal ultrasound arterial signals resemble the signal obtained when cerebral veins are insonated.

Typically the ultrasound power is reduced to 2 MHz or less in order to identify the abnormal ultrasound arterial signals.

According to a third aspect of the present invention there is provided a method of locating vessel occlusion in a target area of a patient, the method comprising transmitting an ultrasound signal into the target area, detecting the signal when returned and determining from the signal whether the vessel is occluded.

Preferably the vessels are small blood vessels.

Alternatively the vessels are large blood vessels.

The method of the second aspect of the present invention is used to determine whether the vessel is occluded.

According to a fourth aspect of the present invention, there is provided a method of screening for small vessel occlusive disease and conditions using the method of the second and third aspects of the present invention.

The disease may be vascular alzheimers. The disease may alternatively be CJD (Creutzfeldt-Jakob Disease). The disease or condition may alternatively be ischaemic stroke, intracerebral hemorrhage, intracerebral tumour, ME, amnesia, irritable bowel syndrome or syndrome X.

According to a fifth aspect of the present invention there is provided a method of treating the symptoms of vessel disease using ultrasound insonation.

Preferably the vessels are small blood vessels. The method can be used to treat all types of small vessel occlusion, for example in the brain, peripheries and also in the retina.

Alternatively the vessels are large blood vessels.

Preferably the vessel disease is identified using the method of the first and second aspects.

The disease may be vascular alzheimers. The disease may alternatively be CJD (Creutzfeldt-Jakob Disease). The disease or condition may alternatively be ischaemic stroke, intracerebral hemorrhage, intracerebral tumour, ME, amnesia, irritable bowel syndrome or syndrome X.

Preferably insonation is carried out using a diagnostic transcranial Doppler ultrasound machine.

Preferably ultrasound insonation is carried out using a 2 MHz probe.

Preferably insonation is continued until the vessel opens or changes in the signals occur. Insonation may be carried out at 100 Mwatts.

Opening of the vessel is identified by changes in the abnormal ultrasound arterial signals present in the second aspect of the present invention. Typically a black area or insonation window appears in the high intensity abnormal arterial signals.

Typically the spectra of the abnormal ultrasound arterial signals changes and the signals become less intense and change from white to red on the doppler ultrasound scan. Typically a low intensity waveform appears super-imposed on the high intensity area.

According to a sixth aspect of the present invention there is provided a method of treating the symptoms of vessel occlusion in a patient using doppler ultrasonography, the method comprising the steps of:
(a) Identifying vessel occlusion in the patient using the methods of the first, second and third aspect of the present invention;
(b) Continuing insonation of the appropriate vessel until changes in the abnormal ultrasound arterial signals are observed. Typically a black area or insonation window appears in the high intensity abnormal arterial signals.

In the step that insonation of the appropriate vessel is continued until the abnormal arterial signals change, insonation is typically carried out at a high frequency. This may be 100 Mwatts or more.

Typically the spectra of the abnormal ultrasound arterial signals changes and the signals become less intense and change from white to red on the doppler ultrasound scan.

Typically a low intensity waveform appears super-imposed on the high intensity area.

According to a seventh aspect of the present invention there is provided a method of treating the symptoms of stroke using transcranial doppler ultrasonography, the method comprising the steps of:
(a) establishing a clinical diagnosis of stroke;
(b) identifying abnormal arterial signals in the appropriate intracerebral artery using the methods of the first, second and third aspects; and
(c) insonating the appropriate intracerebral until changes in the abnormal ultrasound arterial signals are observed. Typically a black area or insonation window appears in the high intensity abnormal arterial signals.

Typically the spectra of the abnormal ultrasound arterial signals changes and the signals become less intense and change from white to red on the doppler ultrasound scan.

Typically a low intensity waveform appears super-imposed on the high intensity area.

The method may include the additional step of carrying out a CT scan after the clinical diagnosis has been established, to determine whether an established infarct is present. Preferably following insonation of the abnormal artery, the patient is monitored for clinical benefit.

According to an eighth aspect of the present invention there is provided a method of ultrasound thrombolysis the method comprising the steps of targeting ultrasound insonation to an area of vessel occlusion on a patient and carrying out prolonged insonation until recanalisation of the vessels occurs.

Preferably the vessels are small blood vessels.

Alternatively the vessels are large blood vessels.

Preferably insonation is conducted at a frequency of at least 100 Mwatts (or maximum power on DWL machine 135 mWatts). Typically insonation can be carried out a frequency up to 200 Mwatts.

Preferably identification of the area of vessel occlusion is carried out by the identification of abnormal arterial ultrasound signals, as described in the first, second and third aspects of the present invention.

Preferably recanalisation of the vessels is identified by the disappearance of abnormal arterial ultrasound signals, as described in the fifth aspect of the present invention.

According to a ninth aspect of the present invention, there is provided a computer program comprising program instructions which, when loaded into a computer, constitute the method of diagnosing vessel occlusion and treating the symptoms of stroke, according to the first to eighth aspects of the present invention.

It will now be described by way of example only an embodiment of the invention, with reference to the following drawings of which:

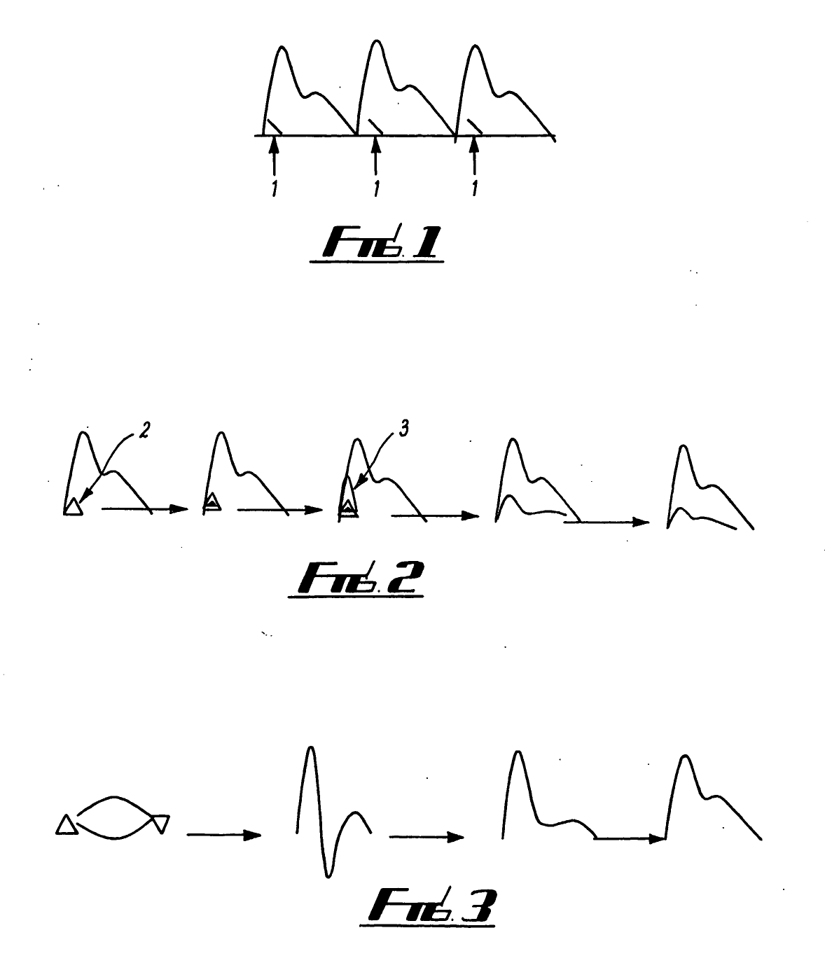

FIG. 1 shows an example of the signal obtained using ultrasound technology for detecting small vessel occlusion. In the present Application this is referred to as “small vessel arterial knock”;

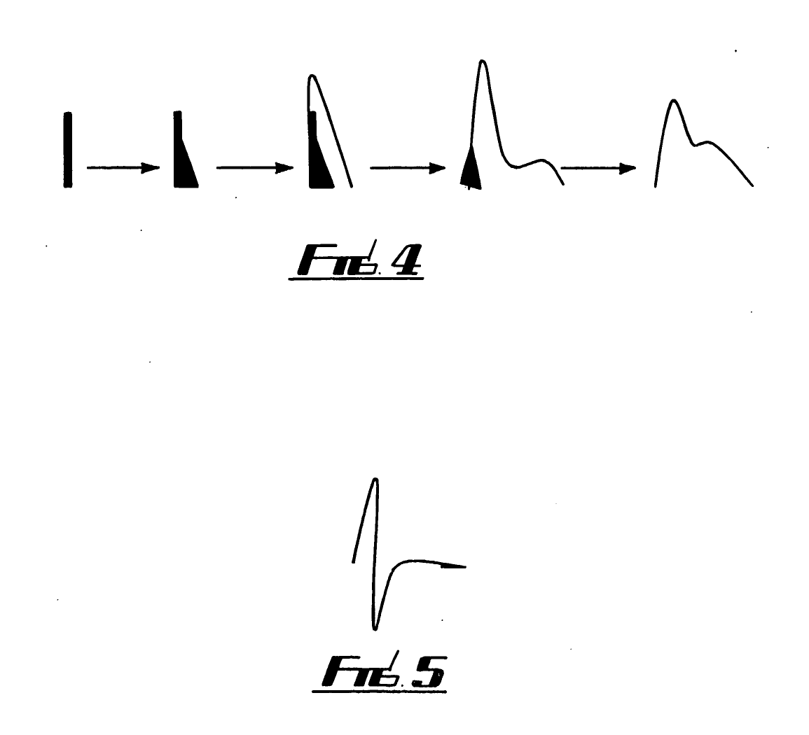

FIG. 2 shows the effect on the signal of insonation on small vessel arterial knock;

FIG. 3 illustrates the arterial knock signal visible using ultrasonography during larger vessel occlusion;

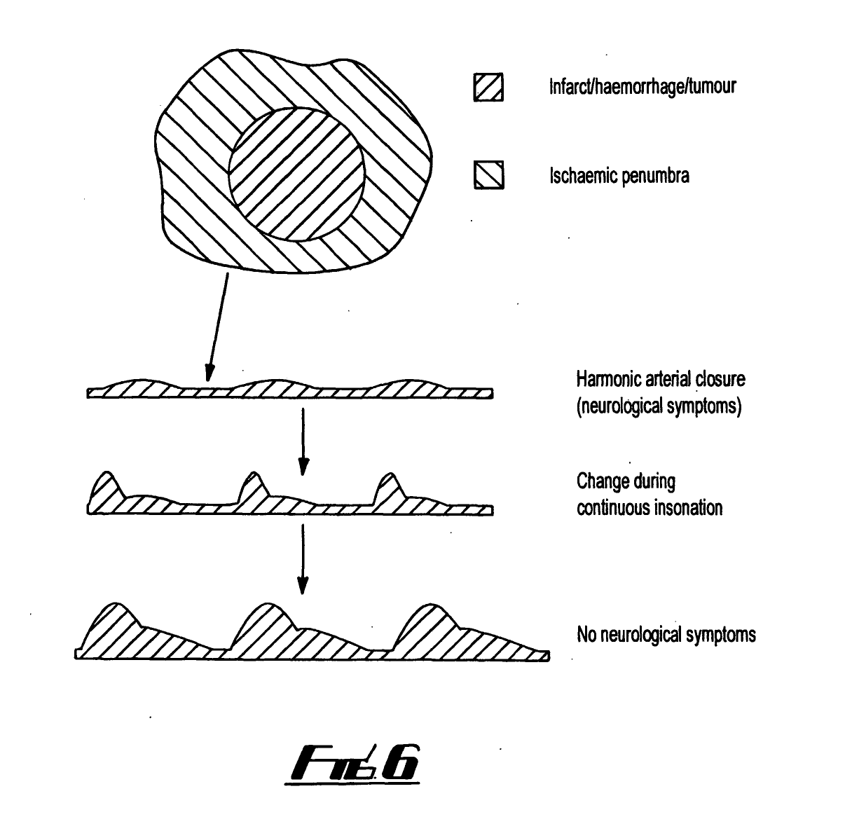

FIG. 4 illustrates occlusion of moderate branches;

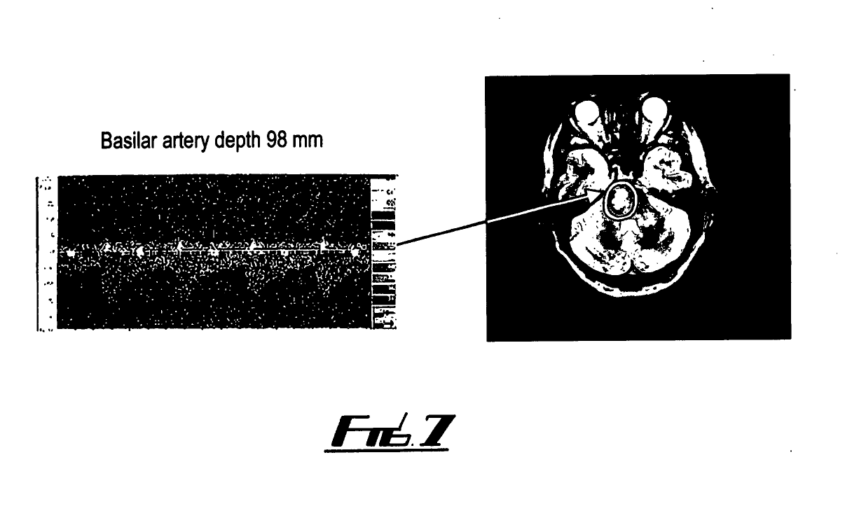

FIG. 5 shows an example signal obtained from distal occlusion of yet larger vessels;

FIG. 6 shows the effect of ultrasound on harmonic arterial closure;

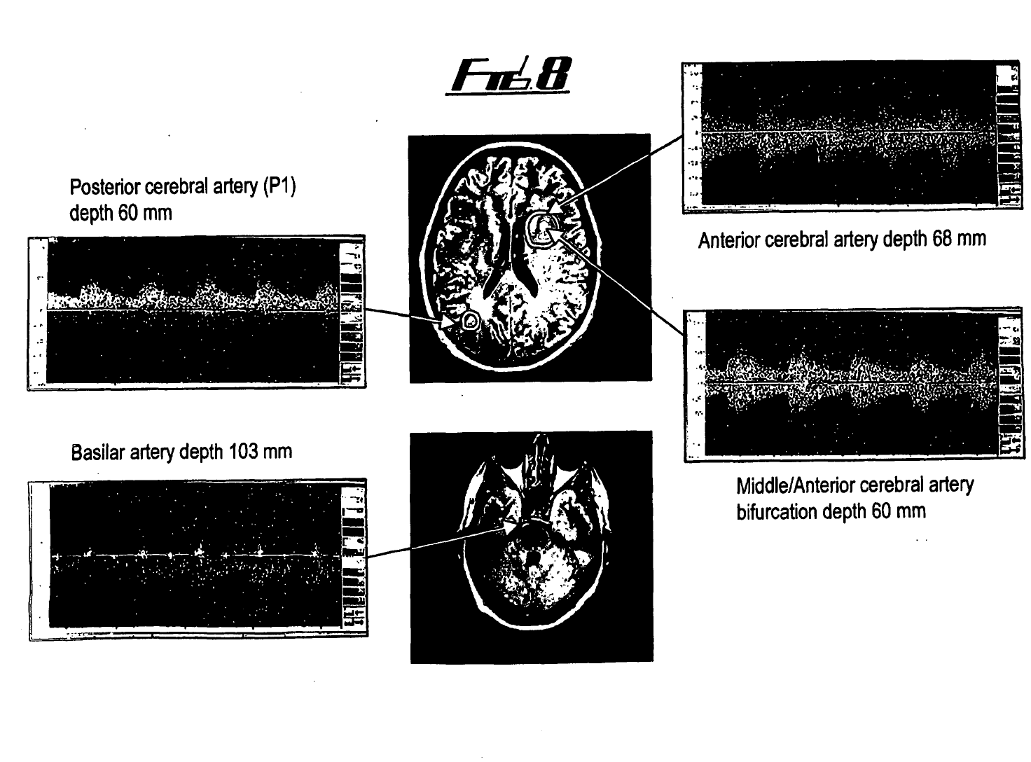

FIG. 7 shows transcranial Doppler ultrasound (TCD) and MRI images from Example 8 described below;

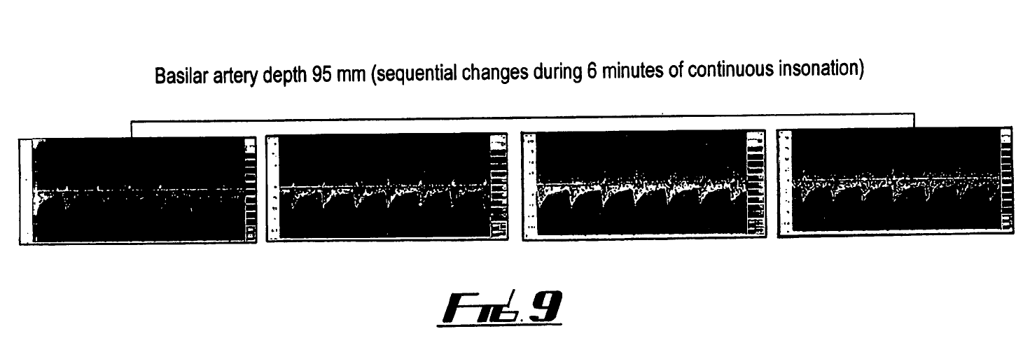

FIG. 8 shows transcranial Doppler ultrasound (TCD) and MRI images from Example 9 described below; and

FIG. 9 shows transcranial Doppler ultrasound (TCD) and MRI images from Example 10 described below.

In the present Application, all reference to research is entirely attributable to Dr Paul Syme, who is the inventor.

The inventor's current research indicates that transcranial Doppler ultrasound scanning can detect small vessel occlusion and can be used as a non-invasive method of therapy on its own for all forms of small vessel ischaemia including all sub-types of ischaemic stroke, ischaemia secondary to primary intracerebral hemorrhage and intracerebral tumour. In addition, the technique herein described can be used to treat all types of small vessel occlusion, for example in the brain, peripheries and also at the back of the retina. The discovery of ultrasound as a diagnostic tool is of particular benefit as small vessels are generally too small to allow accurate visualisation on CAT or MRI scans.

It is common for most TCD machines to use a 300 Hz filter around the baseline in order to eliminate noise at this level. In contrast, it has been discovered in the present invention that removing this filter allows the herein described signals to be obtained, and this allows the hereindescribed techniques and methods to be carried out.

Using the methods described herein the inventor has identified a new Transcranial Doppler ultrasonography (TCD) finding in ischaemic stroke and small vessel occlusion in general, which has been named “small vessel knock”. In the present Application references to “small vessel knock” or “small vessel arterial knock” refer to the discovery of signals that are obtained from small vessel occlusion using targeted transcranial Doppler insonation therapy. These signals occur in small blood vessels and resemble the “knock”, i.e. the short peak systolic wave and diastolic reversal of flow found in circulatory arrest due to brain death⁹. The signal is visible because the sound gets immediately reflected from the blocked vessel and is high intensity low velocity noise. The high intensity is important to the technique, as described below. Small vessel knock is normally biphasic. The signal is visible on the ultrasound scan at systole, typically as a “triangle”. In addition a smaller inverted triangle is nearly always seen at diastole.

The knock is also associated with each cardiac cycle as illustrated in FIG. 1. Small vessel arterial knock can be distinguished from noise, because the high intensity, low velocity signal can be seen at the beginning of each systole (1). This is likely to be lenticulostriate arteries at 80:200 μm in diameter.

Small vessel knock signals are found in small vessel occlusion but knock of a different appearance can also be found in association with large vessel occlusion (line or positive and negative spectra). In this case, the small vessel knock can be large enough to produce a thick line, which appears vertically across the scan and is dependent on cosine theta (cosine of angle between the Doppler sound beam and the axis of blood flow being sampled).

The inventor's current research has also identified a further ultrasonographic finding that will herein be referred to harmonic arterial closure (HAC). This is associated with larger vessels. Demchuk et al. have already classified ultrasound findings in large vessel occlusion. This is called the thrombolysis in brain ischaemia classification (TIBI) and applies to large vessel occlusion only. TIBI is graded from 0 (absent), 1-minimal signal, 2—blunted (systolic peaks only of variable size) 3—dampened (normal systolic and diastolic components seen but reduced) 4-stenotic signal (low intensity high velocity signal caused by stenosis looks like vasospasm). HAC is completely different from these signals. It is found at the baseline like a minimal signal but it resembles a first harmonic signal. It is smooth and is not irregular (blunted). It has a characteristic low pitched humming sound and is a high intensity (blunted signals are low intensity normally) low velocity signal found in association with multiple different pathologies (such as hemorrhage, intracerebral hemorrhage, infarct, tumour, migrainous stroke). HAC opening is normally very quick with the exception of HAC associated with a recent hemorrhage. In this situation the artery opens and then tends to close again quickly. HAC differs from TIBI as it is not sinusoidal, and is entirely positive, as well as being smooth like a first harmonic.

Opening of harmonic arterial closure results in recovery but the timing is important. The work herein described has led to the theory that harmonic arterial closure forms part of large vessel ischaemic penumbra and is likely to be a protective mechanism. The existence of harmonic arterial closure also would suggest that using a non-targeted approach to vessel opening could be dangerous (for example prior efforts using echocontrast with TRUMBI doppler in combination with tPA showed increased hemorrhage).

The key feature in aiding identification (whether of distinct knocks or small harmonic traces caused by harmonic occlusions) is that the impulses (or reflections) from the blockages have a signal intensity which varies according to the rhythm of the patient's heartbeat. Small vessel knock is maximum at peak systole in the cardiac cycle whilst harmonic arterial closure is observed to increase slowly and smoothly across the cardiac cycle.

The high intensity of both small vessel knock and harmonic arterial closure is important for detection as is the lack of a 300 Hz filter since both small vessel knock and harmonial arterial closure are found at the baseline within the +/−300 Hz range. The technique requires that the sound is targeted on to the small vessel knock and harmonial arterial closure. In order to do this, the operator looks for the characteristic signal at the beginning of systole in the main blood vessel. The small vessel knock and harmonial arterial closure are often hidden in the main spectra. Therefore the power is turned down to the lowest setting in order to reveal the small vessel knock and harmonial arterial closure which is camouflaged and drowned out by the main spectral image. Usually the power is adjusted to 2 MHz or less. The position of the probe is then altered to obtain maximal small vessel knock and harmonial arterial closure signals. The probe is then fixed in position and the power turned up to a maximum—usually over 100 Mwatts. At intervals the power is turned down to see the changes to the small vessel knock and harmonial arterial closure. On occasions the small vessel knock and harmonial arterial closure can be seen without reducing the power but in most occasions this is essential to the technique.

Targeting the appropriate vessel at the start of the procedure is also very important and requires a knowledge of the clinical vascular stroke syndrome and a detailed knowledge of the vascular anatomy. Visualisation of the vessels would not help (TCCS machines) in this since small vessel knock is found in small vessels which are MRI, MRA negative and current ultrasound imaging which is based on large vessel detection would not aid small vessel knock and harmonial arterial closure detections.

In the present invention it is shown that HAC is extremely sensitive to ultrasound and that the artery opens within minutes. Using this method the Applicant has shown that stroke secondary to small vessel occlusion can successfully be treated months (although the extent of time over which treatment can occur is currently unknown) after the onset of symptoms, provided MRI and CT scans are megative. This suggests that ischaemic penumbra for small vessel occlusion lasts as long as collateral blood supply is adequate. In other words the brain must be “alive” for the technique to work. A low diagnostic frequency of maximum 2 MHz is used, providing deep penetration, but without the side effect of heat generation. This is particularly advantageous where intracerebral therapy is involved. In the peripheries a larger frequency may be used. As the energy being imparted is typically small, it is expected that the therapy does not act directly on the blood clot, but rather acts on the endothelium to release thrombolytic and vasodilatory agents. It is expected that the technique herein described acts by a mechanical process. Large vessel occlusion can also be treated within 24 hours of onset with clinical improvement but this also requires targeting small vessel branches of the larger vessel. Harmonic arterial closure associated with intracerebral tumour can be reversed months after the onset of symptoms. Targeting ultrasound therapy to small vessels results in opening of these arteries and this is associated with clinical recovery which can in some cases result in complete recovery during insonation. This technique has been successful in all sub-types of ischaemic stroke, intracerebral hemorrhage, vascular closure associated with intracerebral tumour, and has restored memory when the anterior cerebral artery was targeted in innominate stenosis, likely to be diffuse hypoperfusion. The inventor has discovered that amnesia is associated with small vessel knock in the posterior circulation, and has identified a case where small vessel knock is present in transient global ischaemia in the posterior circulation. This suggests the first potential treatment for vascular alzheimers (currrently estimated to be around 40% of all dementia). This technique appears to have no side effects and can be administered safely in the presence of intracerebral hemorrhage.

Referring now FIG. 2, once occlusion has been diagnosed, small vessel arterial knock changes during continued insonation by ultrasound at high frequency (this may be typically in the region of 100 Mwatts or above). The signal becomes less intense (white to red), broadens and a black area appears in the original high intensity signal. The black area often looks like a triangle on the white reflected sound and can be multiple. This has been termed as the insonation window by the inventor, and occurs as there is little or no reflection of the signal back. A low intensity waveform can be seen super-imposed on the high intensity area, often of high velocity as the artery opens. This change is always associated with clinical recovery to some extent. This low intensity waveform increases in intensity and a diastolic component of this waveform then appears (in FIG. 2 triangles are enlarged for diagrammatic purposes). High intensity, low velocity small vessel arterial knock is illustrated at 2 in FIG. 2, whilst low intensity high velocity signal with no diastolic component is illustrated at 3.

Referring to FIG. 3, the larger more obvious systolic peak can be seen with the smaller less obvious diastolic peak.

When moderate to larger branches, for example 200 μm upwards, occlude, this can appear as an obvious line in the spectra, as shown in FIG. 4. Generally speaking smaller vessels produce a smaller knock whilst larger vessels produce a larger, and more obvious knock. Vessels with infarct already established are resistant to opening. Distal occlusion of even larger vessels for example 400 μm upwards, is shown in FIG. 5 with the forward flow equal to reverse flow. Increased arterial flow occurs during ultrasound insonation and this is likely to be due to arterial dilatation. Thus, insonation results in both clot lysis and increased blood flow. The mechanism by which ultrasound does this is unknown. However, there is evidence that sheer stress to endothelium results in the release of local tPA, thrombomodulin which binds thrombin and the release of nitric oxide which is a potent vasodilator.⁸ Since it is likely that the mechanism by which ultrasound opens blood vessels will be universal, this technique will be applicable to any tissue in the body where small vessel ischaemia exists and may have application in, for example, graft rejection, kidney damage, retinal artery occlusion, brain or heart disease.

Large vessel (>800 μm diameter) occlusion also responds to insonation. The ultrasound appearances have already been described. However, the branches of the large vessel M1, M2 of the middle cerebral artery and A1 of the anterior cerebral artery then need to be identified in space and insonated up and down the artery to a depth of around 30 mm from the surface whenever possible otherwise no recovery occurs.

Harmonic arterial closure is very sensitive to ultrasound. This arterial signal is found in migraine, all injury infarct and in association with intracerebral hemorrhage with ischaemic stroke and with intracerebral tumour. This abnormal artery opens rapidly with insonation. The inventor believes this is the mechanism by which the brain protects itself from damage and is part of the large vessel “ischaemic penumbra” as shown in FIG. 6. Opening these vessels without restoring blood flow from occluded vessels could be dangerous and requires a targeted approach to therapy.

Opening of small vessel knock with recovery can occur over months, which implies that the ischaemic penumbra for small vessel occlusion lasts as long as the collateral blood supply can protect the endangered brain tissue. A positive MRI result suggests cytotoxic oedema which only occurs when death of tissue is imminent or has already occurred. Small vessel knock with symptoms with normal MRI implies that a full recovery is possible at any stage if the vessel can be opened. However large vessel occlusion always will result in damage. This may explain why opening large vessels with sound has so far not resulted in detecable recovery.

It has been discovered that large vessel TIBI occlusion, harmonic arterial closure and small vessel knock can exist together in the same patient.

The method of the present invention uses a diagnostic Transcranial Doppler ultrasound machine (such as Ezdop DWL or Spencer Technology headset) and is carried out after clinical diagnosis of stroke is established using, for example, the following criteria; assessment of symptoms, sudden in onset, focal as compared to global neurological symptoms and signs, no other cause other than stroke, likely to be a particular arterial territory. A CT scan and MRI should also be performed whenever possible to determine whether an established infarct is already present, whether the focal neurology is due to hemorrhage (i.e., to exclude possibility of hemorrhage), or whether the signs and symptoms are due to tumour. If an infarct is already established for the targeted artery then opening this artery with ultrasound is of limited benefit. Hypoperfusion suggesting early infarction does benefit form insonation. The CT is only a guide to established infarction or extensive hemorrhage but is not necessary prior to insonation. Using these clinical methods, the area of occlusion can be identified. A diagnostic software program may also be used at this stage to allow computer aided identification of the area.

The diagnostic and therapeutic method of the present invention can be carried out as follows:
(a) Identification of the appropriate intracerebral artery is carried out using clinical methods such as assessment of symptoms and knowledge of the vascular anatomy. Abnormal arterial signals (small vessel arterial knock, large vessel branch occlusion and harmonic arterial closure as described above) are identified using Doppler ultrasound scanning in the appropriate intracerebral artery (as illustrated in the Figures) using visual and audible signals in the manner described above. The ultrasound power is typically reduced to 2 MHz or less so that the signals can be detected around the baseline. Once detected the probe is fixed and power turned up to high frequency.
(b) Insonation of the abnormal artery is continued on high power (e.g., often in the region of 100 Mwatts and above) until the artery opens or the systolic triangular signal changes and the insonation window appears. The duration of insonation prior to opening varies. Harmonic arterial closure opens rapidly in less than 5 minutes. Small vessel arterial knock takes around 15 minutes. Knock from larger vessels is resistant to opening but recent large vessel occlusion opens around 15 to 20 minutes.
(c) Asking the patient whether there is any clinical benefit. This helps to direct the operator to the correct artery and insonation angle. The technique is blind in that no arterial image other than the Doppler signal is obtained. However, targeting abnormal arteries does not require the patient to be conscious.
(d) Collating the above in a clinical algorithm and the detection of abnormal images aided by computer.

Full recovery tends to occur if the vessel fully opens and full opening of the artery tends to result in no recurrence. However recurrence of the occlusion or closure can occur in some cases. In particular if the end result is an insonation window (black area within the white triangle) the recurrence tend to occur. It is postulated that this occurs as there is still a partial occlusion, and as a result the patient has symptoms upon standing. However, these recurrences respond again to insonation. Nevertheless, vessels with harmonic arterial closure in hemorrhage have been found in some cases to be resistant to opening and may only show a transient opening. It will also be appreciated that the method herein described is an acute treatment and doe not negate the need for secondary prevention.

This technique is applicable to both ischaemic and haemorrhagic stroke. Patients with the same vessel abnormalities secondary to tumour will also benefit from the above technique. The technique has further applications in other types of small vessel disease, such as heart disease, retinal artery occlusion, graft rejection, kidney disease, etc.

Small vessel arterial knock has not previously been described in relation to stroke. It is common for most TCD machines to use a 300 Hz filter around the baseline in order to eliminate noise at this level. In contrast, it has been discovered in the present invention that removing this filter allows the herein described signal to be obtained. The signal varies from a small triangular noise to a line. The larger the line and noise the more resistant the artery is to opening. However, the abnormal signal can also be a bruit and the knock is normally biphasic. Generally the systolic component of the knock can be seen, however a diastolic component is nearly always also observed. Small vessel knock can also appear as a large reflected sound line going right across the screen vertically through the small vessel knock. This occurs when the sound hits the small vessel knock head on. When the sound crosses a branch sometimes a false-positive small vessel knock can be detected but these do not change with insonation and insonation without change to a small vessel knock-like piece of noise does not result in any recovery.

Small vessel knock can be detected in the anterior cerebral circulation (middle cerebral, anterior cerebral artery territories) and also the posterior circulation territories (vertebral arteries, basilar arteries). The Applicant has shown that using the method of the present invention, insonating the knock results in clinical recovery.

Advantageously the ultrasonography technique described in the present Application uses a low frequency (2 MHz or below), and therefore generates little heat.

Eleven cases are detailed below which provide evidence of spontaneous recanalisation during TCD insonation. This was associated with clinical recovery.

The following relate to large vessel occlusion.

### EXAMPLE 1

Example 1 was a 45 year old man who presented with sudden onset of a dense hypotonic right hemiplegia with expressive dysphasia. This resulted from occlusion of his right internal carotid artery in the neck. Insonation was 2 hours post-onset. During insonation there was some return of power to his right side. His dysphasia improved over the next few hours. This clinical situation persisted for 48 hours, but then his dense right hemiplegia returned. TCD insonation at 72 hours showed that the left MCA has reoccluded. A repeat CT scan showed a moderate right NCA infarct. The CT scan 2 hours post-onset showed the left middle cerebral artery (MCA) hyperdensity sign and at a 72 hours a moderate infarct. At the start of insonation no flow was obtained in the left MCA, but during continuous insonation this appeared and then increased in intensity over a period of 20 minutes. He had evidence of both anti-rear and left posterior communicating artery flow consistent with an intact circle of willis.

### EXAMPLE 2

Example 2 was a 55 year old woman who presented with the sudden onset of a dense hypotonic left hemiplegia with severe inattention. This resulted from occlusion of her right internal carotid in the neck. Insonation was commenced 2 hours post-onset. This patient recovered full power after 40 minutes of continuous TCD insonation. Recovery was associated with the opening of the right MCA. On reocclusion hemiplegia returned and persisted despite obtaining a stenotic flow with further insonation.

### EXAMPLE 3

Example 3 was a 56 year old man who had an aneurysm of his heart and a tight stenosis of right internal carotid artery, who presented with a complete right hypotonic hemiplegia and aphasia. This patient was insonated at 48 hours. Following insonation there was no improvement in either his hemiplegia or aphasia, but he became less drowsy. An MI occlusion of the left middle cerebral artery was identified. Initially there was no visible signal from the left MCA, but this again appeared and increased in flow during continuous insonation over a period of 20 minutes. His CT prior to insonation showed that a large left MCA infarct was already established.

### EXAMPLE 4

Example 4 is a 40 year old man who presented with a sudden onset of weakness on the right hand side 48 hours after hip replacement. Complete dysphasia and paralysis had been present for 12 hours. Evidence of 0-4 TIBI was present in the left MCA, together with knock in the form of a straight line (as described above) in relation to TIBI. Insonation performed up and down arteries resulted in clinical recovery and recovery of speech. Patent foramen ovale was identified as the cause of a paradoxical embolic event.

There follows three examples of Harmonic Arterial Closure. The inventor has identified the crucial factor that in these cases the arteries open within a couple of minutes and the signal is NOT blunted (and is thus different from the Thrombolysis in brain ischaemia (TIBI) 1-3 seen in large vessel occlusion) but extremely smooth like a first harmonic.

### EXAMPLE 5

Example 5 is that of a 36 year old woman with a history of migraine. She developed sudden onset of numbness of her left arm, hand and leg. This has persisted for 48 hours prior to insonation. During 20 minutes of insonation, this paraesthesia completely resolved. CT, echocardiography and carotid duplex were all normal. Using the herein described method, abnormal flow was identified in a branch of the right MCA. This flow improved over 20 minutes of continuous insonation. Her CT scan was normal.

### EXAMPLE 6

Example 6 was that of a 51 year old male who presented whilst out running with sudden onset of a mild left sided hemiplegia, reduced sensation and slurred speech. This situation persisted for the next 48 hours, during which time he mobilised independently. He then developed sudden onset on a dense weakness of his left leg with moderate weakness of his left arm, associated with complete paraesthesia of the leg and reduced sensation in the arm. TCD insonation was performed 25 minutes after the onset of the second episode. During 20 minutes of insonation his power and sensation completely returned to that found on admission. This patient has had no reoccurrences over the past 6 months. It was seen that all of the main blood vessels were open, there was an increased pulsability index in the right MCA, compared with the left MCA. TCD findings 25 minutes after the onset of the new episode of dense hemiplegia showed an abnormal signal consistent with arterial near occlusion in a small branch of the right middle cerebral artery. During 20 minutes of continuous insonation, the flow in this branch increased. The initial CT scan taken prior to insonation showed a right basal ganglia hemorrhage. The second CT scan performed post-insonation showed that the cerebral hemorrhage had not increased in size between scans.

### EXAMPLE 7

Example 7 is a 45 year old lecturer who presented with dysphasia following dissection of his left internal carotid artery and infarct. Using the herein described method two vessels with harmonic arterial closure were identified in the left MCA territory. Insonation opened these and resulted in a marked improvement in speech.

All of the abovementioned methods used a 2 MHz probe for TCD (Ezdop DWL) via a transtemporal window. Prolonged insonation was performed at 100 mW. These cases provide evidence that clinical recovery is associated with opening of abnormal arteries during continuous transcranial Doppler insonation alone, and without the necessity to administer, for example, TPA.

In the three described cases of main MCA occlusion, two were secondary to internal carotid artery occlusion, and one to cardio-embolism. In the descried cases of MCA branch occlusion, one was due to a primary intracerebral hemorrhage, one to migraine and one to ischaemia. The time of TCD post-stroke varied from 25 minutes to 48 hours. The results of TCD were that the MCA opened within 20 minutes in four cases, and 40 minutes in one (absent posterior communicator). In all cases, opening of the artery was associated with clinical improvement. Reocclusion occurred in the two cases of ICA occlusion, resulting in hemiplegia in one, and death in another. Benefit was obtained following recanalisation, even at 48 hours. These cases give further support to the therapeutic potential TCD and, in particular, the case of recanalisation of an occluded MCA branch at the site of PIH during TCD is unique, and provides evidence for PIH induced ischaemia due to local arterial tamponade.

The following cases show that TCD can detect SVD in the form of “small vessel knock” in patients with MRI positive and negative stroke-like deficits.¹⁰ Insonation can open these occlusions resulting in clinical improvement (with a large therapeutic window) if MRI-negative. The mechanism of action has to be physical. Ultrasound may simulate endothelial flow stress releasing endogenous tPA¹¹ and nitric oxide.¹²

### EXAMPLE 8

Referring to FIG. 7, Example 8 is a 67 year old man who presented with sudden onset of left face, arm and leg weakness with mild dysarthria. A T2-weighted MRI slice through the pons showed a hyperintensity signal consistent with an infarct. TCD performed 12 hours post-onset showed an abnormal high intensity low velocity signal occurring at peak systole with an inverted signal during diastole, to the right of the main basilar artery, at a depth of 103 mm. Continuous insonation improved flow (not shown) but did not result in any recovery.

### EXAMPLE 9

Referring to FIG. 8, Example 9 is a 44 year old women with a 7 week history of intermittent, left sided weakness, dizziness and mild paraesthesia. The figure shows two FLAIR MRI slices, one with left basal ganglia hyperintensity signals consistent with small vessel occlusive disease (SVD). These signals were associated with TCD SVK in the left anterior cerebral artery (ACA), the posterior cerebral artery and noise at the ACA/middle cerebral artery junction. This patient also had SVK to the right of the basilar artery as per Case 1 with normal brain-stem MRI. Prior to insonation she had been symptomatic for over 48 hours. Continuous insonation of the basilar SVK improved flow and relieved her symptoms.

### EXAMPLE 10

Referring to FIG. 9, Example 10 is an 86 year old women who presented with sudden onset of left-sided facial pain associated with paraesthesia. Her pattern of allodynia was consistent with a trigeminal neuropathy. TCD performed after 6 weeks of symptoms identified SVK in the basilar territory and continuous insonation resulted in improvement of flow (see Figure). This was associated with a return of normal sensation to her face. MRI of the brain-stem was also normal.

### EXAMPLE 11

Example 11 is a 79 year old retired engineer who had a sudden onset of balance problems and was found to have Small vessel knock in the L vertebral artery. Insonation opened this Small vessel knock and improved his symptoms. However, this patient over the next few months continued to deteriorate and on questioning appeared to have had memory problems prior to the sudden loss of balance. The memory problem continued to worsen. An MRI suggested widespread small vessel occlusion consistent with vascular Alzheimers. However, Transcranial doppler ultrasonography did not reveal small vessel knock in the relevant arterial territories. An autopsy was performed and this showed sporadic CJD and NOT small vessel occlusion confirming the negative Transcranial doppler ultrasonography findings. This case emphasises the specificity of Transcranial doppler ultrasonography small vessel knock detection. Thus, the Syme Insonation Technique™ of small vessel knock detection is not only the most sensitive technique for detecting small vessel occlusion but is more specific for this than MRI.

The work carried out by the inventor has also led to the theory that small vessel knock is the cause in some cases of sudden onset trigeminal neurlagia and neuropathy and cluster headaches. Small vessel knock can cause Dejerrines Syndrome (Medial medullary syndrome), lateral medullary syndrome (PICA and Opalski syndrome) and is found in Syndrome X (atypical chest pain with normal coronary arteries). Transient global amnesia is also associated with small vessel knock but with an insonation window (black triangle) in the knock. This is the feature found in knock following insonation (as descibed above) and is always associated with recovery. This suggests that Small vessel knock is important for amnesia and this technique could be used to treat amnesia associated with vascular Alzheimers (40% of dementia). The small vessel knock can be found in MRI positive and negative cases and thus the technique could be used to screen individuals. Small vessel knock has been observed on both sides of the brain in ME and identified in Syndrome X and irritable bowel syndrome.

In the present invention, an abnormal arterial signal similar to the arterial systolic knock found in circulatory arrest associated with brain death, has been found at peak systole within 300 Hz of the baseline. It is possible that small vessel knock has not been previously reported because the first 300 Hz of most TCD machines are normally automaticallly filtered to remove spectral noise. Small vessel knock identification allows the prospect of early Transcrannial Doppler Ultrasonography detection of small vessel occlusion in MRI-negative stroke.

The method and technique of the present invention is successful in isolation to other therapies, and would therefore appear to offer a non-invasive effective treatment for all sub-types of stroke.

The method herein described may also be used for screening for small vessel occlusive disease. Non invasive screening for diseases such as vascular Alzheimers and CJD (Creutzfeldt-Jakob Disease) is envisaged using the described technique.

It should be noted that the embodiments disclosed above are merely exemplary of the invention, which may be embodied in different forms. Therefore details disclosed herein are not to be interpreted as limiting, but merely as a basis for claims and for teaching one skilled in the art as to the various uses of the present invention in any appropriate manner.

### Documents Referenced Herein

The citations referred to above are included within this disclosure by way of this reference.
1. Anonymous. Effect of intravenous recombinant tissue plasminogen activator on ischaemic stroke lesion size measured by computed tomography. NINDS; The National Institute of Neurological Disorders and Stroke (NINDS) rt-PA Stroke Study Group. Stroke. 2000; 31(12):2912-9.
2. Hacke W, Kaste M, Fieschi C, et al.—Intravenous thrombolysis with recombinant tissue plasminogen activator for acute hemispheric stroke. The European Cooperative Acute Stroke Study (ECASS) [see comments]. (13):1017-1255.
3. Hong H, Liu GQ. Current status and perspectives on the development of neuroprotectants for ischaemic cerebrovascular disease. Drugs of Today; 39(3):213-22.
4. El-Mitwalli A, Saad M, Christou I, Malkoff M, Alexandrov A V. Clinical and sonographic patterns of tandem internal carotid artery/middle cerebral artery occlusion in tissue plasminogen activator-treated patients. Stroke. 2002; 33(1):99-102.
5. Ishibashi T, Akiyama M, Onoue H, Abe T, Furuhata H. Can Transcranial Ultrasonication Increase Recanalization Flow With Tissue Plasminogen Activator? Stroke 2002; 33(5):1399-1404.
6. Cintas P, Le Traon A P, Larrue V. High Rate of Recanalization of Middle Cerebral Artery Occlusion During 2-MHz Transcranial Color-Coded Doppler Continuous Monitoring Without Thrombolytic Drug. Stroke 2002; 33(2):626-628.
7. Labiche L A, Al-Senani F, Wojner A W, Grotta J C, Malkoff M, Alexandrov A V. Is the Benefit of Early Recanalization Sustained at 3 Months? A Prospective Cohort Study. Stroke 2003:01.5TR.0000055940.00316.6B.
8. Diamond S L, Eskin S G, McIntire L V. Fluid flow stimulates tissue plasminogen activator secretion by cultured human endothelial cells. Science. 1989; 243 (4897):1483-5.
9. Wijdicks E F. The diagnosis of brain death. N Engi J. Med. 2001; 344:1215-1221.
10. Ay H, Buonanno F S, Rordorf G, Schaefer P W, Schwamm L H, Wu O, Gonzalez R G, Yamada K, Sorensen G A, Koroshetz W J. Normal diffusion-weighted MRI during stroke-like deficits. Neurology. 1999; 52:1784-1792.
11. Diamond S L, Eskin S G, McIntire L V. Fluid flow stimulates tissue plasminogen activator secretion by cultured human endothelial cells. Science. 1989; 243:1483-1485.
12. Ozawa N, Shichiri M, Iwashina M, Fukai N, Yoshimoto T, Hirata Y. Laminar shear stress up-regulates inducible nitric oxide synthase in the endothelium. Hypertens Res. 2004; 27:93-99.

1. A method of diagnosing vessel occlusion in a patient comprising the steps of:
using transcranial Doppler ultrasonography to identify abnormal ultrasound arterial signals on a Doppler ultrasound scan;
wherein the abnormal ultrasound arterial signals are identified at a baseline frequency of approximately 2 megahertz (MHz) within a range of approximately 300 Hz above or below the baseline frequency.

2. A method as claimed in claim 1, wherein the abnormal ultrasound arterial signals are high intensity, low velocity.

3. A method as claimed in claim 1 wherein the abnormal ultrasound arterial signals are associated with each cardiac cycle.

4. A method as claimed in claim 1 wherein the abnormal ultrasound arterial signal has an intensity which varies according to the rhythm of the patient's heartbeat.

5. (canceled)

6. (canceled)

7. A method as claimed in claim 1 wherein the vessels are small blood vessels of approximately 80 micrometers in diameter to less than approximately 200 micrometers in diameter.

8. A method as claimed in claim 7, wherein the abnormal ultrasound arterial signals resemble the short peak systolic wave and diastolic reversal of flow which can be seen with circulatory arrest due to brain death.

9. A method as claimed in claim 1, wherein the abnormal ultrasound arterial signals can be seen at the beginning of each systole of the cardiac cycle.

10. A method as claimed in claim 7 wherein the abnormal ultrasound arterial signals also have a diastolic component.

11. A method as claimed in claim 1 wherein the vessels are large blood vessels of approximately equal to or greater than 200 micrometers in diameter.

12. A method as claimed in claim 11, wherein the abnormal ultrasound arterial signals take the form of a first harmonic signal.

13. A method as claimed in claim 1, wherein the abnormal ultrasound arterial signals resemble the signal obtained when cerebral veins are insonated.

14. A method as claimed in claim 11 wherein the abnormal ultrasound arterial signals are accompanied by a low pitched humming sound.

15. A method of screening for small vessel occlusive diseases and conditions, comprising:
diagnosing vessel occlusion using transcranial Doppler ultrasonography to identify abnormal ultrasound arterial signals on a Doppler ultrasound scan;
wherein the abnormal ultrasound arterial signals are identified at a baseline frequency of approximately 2 megahertz (MHz) within a range of approximately 300 Hz above or below the baseline frequency.

16. A method of treating the symptoms of vessel occlusion using ultrasonography, comprising the steps of:
identifying vessel occlusion by detecting abnormal ultrasound arterial signals using ultrasonography at a baseline frequency of approximately 2 MHz within a range of approximately 300 Hz above or below the baseline frequency; and
insonating the identified vessel occlusion to treat the symptoms of vessel occlusion.

17. A method as claimed in claim 16, wherein vessel occlusion is first diagnosed using the method of claim 1.

18. A method as claimed in claim 16 wherein the vessels are small blood vessels of approximately 80 micrometers in diameter to less than approximately 200 micrometers in diameter.

19. A method as claimed in claim 16, wherein the vessels are large blood vessels of approximately equal to or greater than 200 micrometers in diameter.

20. A method as claimed in claim 16 using transcranial Doppler ultrasonography.

21. A method as claimed in claim 16 wherein ultrasound insonation is carried out at a power of at least 100 mwatts.

22. A method as claimed in claim 15 wherein the vessel is insonated until changes in the abnormal ultrasound arterial signals occur.

23. A method as claimed in claim 22, wherein a black area appears in the high intensity abnormal arterial signals in a color spectrogram of the Doppler ultrasound scan.

24. A method as claimed in claim 22, wherein the spectra of the high intensity abnormal arterial signals changes.

25. A method as claimed in claim 22 wherein the high intensity abnormal arterial signals change from white to red on a color spectrogram of the Doppler ultrasound scan.

26. A method of treating the symptoms of vessel occlusion in a patient using Doppler ultrasonography, the method comprising the steps of:
(a) identifying vessel occlusion in the patient using a method of identifying abnormal ultrasound arterial signals on a Doppler ultrasound scan using transcranial Doppler ultrasonography, wherein the abnormal ultrasound arterial signals are identified at a baseline frequency of approximately 2 megahertz (MHz) within a range of 300 Hz above or below the baseline frequency; and
(b) continuing insonation of the appropriate vessel until changes in the abnormal ultrasound arterial signals occur.

27. A method as claimed in claim 26 where in the step that insonation of the appropriate vessel is continued until changes in the abnormal arterial signals occur, insonation is carried out at a power of at least 100 mwatts.

28. A method as claimed in claim 26 where in the step that insonation of the appropriate vessel is continued until changes in the abnormal arterial signals occur, the abnormal arterial signals become less intense and change from white to red on the Doppler ultrasound scan.

29. A method as claimed in claim 26 where in the step that insonation of the appropriate vessel is continued until changes in the abnormal arterial signals occur, the spectra of the high intensity abnormal arterial signals changes.

30. A method as claimed in claim 26 where in the step that insonation of the appropriate vessel is continued until changes in the abnormal arterial signals occur, a black area appears in the high intensity abnormal arterial signals.

31. A method of treating the symptoms of stroke using transcranial Doppler ultrasonography, the method comprising the steps of:
(a) establishing a clinical diagnosis of stroke;
(b) identifying abnormal arterial signals in the appropriate intracerebral artery using a method of identifying abnormal ultrasound arterial signals on a Doppler ultrasound scan using transcranial Doppler ultrasonography, wherein the abnormal ultrasound arterial signals are identified at a baseline frequency of less than approximately 2 megahertz (MHz) within a range of approximately 300 Hz above or below the baseline frequency; and
(c) insonating the appropriate intracerebral artery until the abnormal arterial signals disappear.

32. A method as claimed in claim 31, which includes the additional step of carrying out a CT scan after the clinical diagnosis has been established, to determine whether an established infarct is present.

33. A method as claimed in claim 31, wherein the patient is monitored for clinical benefit following insonation of the abnormal artery.

34. A method of ultrasound thrombolysis, the method comprising the steps of:
targeting ultrasound insonation to an area of vessel occlusion on a patient, wherein the area of vessel occlusion is located by identifying abnormal ultrasound arterial signals on a Doppler ultrasound scan using transcranial Doppler ultrasonography, and wherein the abnormal ultrasound arterial signals are identified at a baseline frequency of approximately 2 megahertz (MHz) within a range of approximately 300 Hz above or below the baseline frequency; and
carrying out prolonged insonation until recanalisation of the vessels occurs.

35. A method as claimed in claim 34, wherein the vessels are small blood vessels of approximately 80 micrometers in diameter to less than approximately 200 micrometers in diameter.

36. A method as claimed in claim 34, wherein the vessels are large blood vessels of approximately equal to or greater than 200 micrometers in diameter.

37. A method as claimed in claim 34 wherein the area of vessel occlusion is located by the identification of abnormal arterial ultrasound signals as claimed in claim 1.

38. A method as claimed in claim 34 wherein recanalisation of the vessel is carried out using ultrasound insonation.

39. A non-transitory computer program product embodied on a computer-readable medium comprising computer code and program instructions which, when loaded into a computer, comprise a method of diagnosing vessel occlusion by:
identifying abnormal ultrasound arterial signals on a Doppler ultrasound scan using transcranial Doppler ultrasonography; wherein the abnormal ultrasound arterial signals are identified at a baseline frequency of approximately 2 megahertz (MHz) within a range of approximately 300 Hz above or below the baseline frequency; and
displaying the abnormal ultrasound arterial signals.
